(19) United States
(12) Patent Application Publication
(10) Pub. No.: US 2002/0198447 A1
(43) Pub. Date: Dec. 26, 2002

(54) Automatic prescription of tomographic imaging parameters

(76) Inventors: Arianne Margarethe Corinne Van Muiswinkel, Eindhoven (NL); Johan Samuel Van Den Brink, Eindhoven (NL)

Correspondence Address: THOMAS M. LUNDIN, Philips Medical Systems (Cleveland), Inc., 595 Miner Road, Cleveland, OH, 44143, US

(21) Appl. No.: 10/150,136

(22) Filed: May 16, 2002

(30) Foreign Application Priority Data
May 16, 2001 (EP) 01201831.3

(51) Int. Cl. A61B 5/05

(52) U.S. Cl. 600/410

(57) ABSTRACT

The invention relates to a method for prescription of scanning parameters determining the orientation and location of tomographic imaging planes. The invention facilitates the process of re-scanning a patient at different times. This is achieved by the computation of the current orientation and location of the patient relative to his or her orientation and location during a previous examination by matching (3) a current reference scan image (1) with the image data of a corresponding previous reference scan (2). The current examination scanning parameters are calculated by adjusting the scanning parameters of the previous examination in accordance with the relative position of the patient during the current examination.

### BACKGROUND

The invention relates to a method for prescription of scanning parameters determining the orientation and location of tomographic imaging planes, wherein a current reference scan of an object is performed, the image data of the reference scan being analyzed, thereby extracting geometric data defining the current orientation and location of the examined object in the scanner, and wherein scanning parameters for one or more current examination scans are computed by relating the current geometric data of the examined object to the corresponding geometric data of the same object during a previous examination.

Furthermore, the invention relates to a computer program for carrying out the method of the invention and a tomographic imaging apparatus operating according to this method.

In medical imaging, such as magnetic resonance imaging (MRI) or computer tomography (CT), an image of a section or slice of a region of interest of a patient is reconstructed from the magnetic resonance signals (MRI) or the X-ray beam projections (CT). Typically, the scanning of patients for medical purposes is performed according to manual prescriptions of the scanning parameters determining the orientation and location of the imaging planes. Because of the variability in the selection of the most appropriate angulation and off-center parameters of the imaging planes it is a difficult task to achieve good reproducibility for scans of the same patient which are repeated at different examination sessions at different times.

There is a need to facilitate the process of re-scanning a patient at different times. This is because there are several groups of patients with diseases, like for example cancer, multiple sclerosis, Alzheimer's disease, and others, which have to be examined several times in order to assess the progression of the disease and the success of the applied therapy. An accurate analysis of the development requires precise prescription of scanning parameters. Furthermore, accurate re-scanning also plays an important role in interventional radiology. Current practice is to send the patient for a repeated tomographic examination with the imaging results of the previous examinations on film. From these images the radiologist or the operator of the tomographic apparatus then usually tries to trace back the scanning parameters of the previous examination. This proceeding is very laborious, cumbersome and little precise, particularly if several angulations of different scans are involved. The totally manual prescription of scanning parameters for re-scanning of a patient thus takes a long time and is not very accurate in terms of positioning of the imaging planes. Often more than one attempt is required before reasonable results are obtained.

Therefore it is readily appreciated that there is a need for methods which enable a more or less automated prescription of scanning parameters for repeated rescanning of the same patient, thereby minimizing the involvement of human operators. Such methods are particularly useful if they directly provide a set of scanning parameters depending on the corresponding parameters of previous examinations.

Such a method is for example described in U.S. Pat. No. 6,195,409 B1. According to this known method one or more so-called localizer scans are performed initially. Thereafter, these localizer images are analyzed in order to extract structural information about the examined object, such as size, location and orientation of the object or organ. As a result of this analysis, an abstract, schematic description (a model) of the examined object is obtained. This abstract schematic description is then matched with a so-called reference template which additionally contains information about the location of the imaging planes and the scanning parameters. The above mentioned model is an abstract, schematic description of the object of interest consisting of geometric and structural information corresponding to geometric and physical attributes of the object. The model comprises for example coordinates identifying characteristic features of the examined object, such as the tip of the nose, an eye and other reference points which correspond to characteristic anatomic structures. Extracting the parameters of the model from the localizer images involves geometric transformations which are iteratively performed until an optimal fit between the generic model and the particular image data is obtained. Once the model is matched with the image data, the various imaging planes are determined from the planes of the template, thus allowing the automated prescription of new scanning parameters.

The main drawback of the above technique is based on the use of the abstract, schematic model. The implementation of the complex matching procedure of the model with the reference image of the examined object is difficult or even not practicable, particularly on tomographic scanners with limited computing capacities. The generic model is most appropriate to automatically select optimal, standard imaging planes for any individual patient, but it is not optimally suited to meet the specific requirements of re-scanning the same patient repeatedly at different examination sessions at different times. This is because the known method makes no use of individual characteristic features of a single individual which can advantageously be exploited if the same examination of the same patient has to be performed again and again. Indeed, it is already current practice of the operators of tomographic scanners to use such individual features to manually trace back the scanning parameters of the previous examinations. In contrast to this, the known method will fail to accurately find the same orientation and location of the imaging planes again if changes of the object of interest occur between the examinations, as it is typically the case during the progression of a disease like cancer, multiple sclerosis and others. In such situations not only the orientation and position of the examined object in the scanner but also its physical properties have changed during the period between the examinations.

### SUMMARY

It is consequently the primary objective of the present invention to provide an improved technique for the prescription of scanning parameters for tomographic imaging.

It is a further object to enable a fully or semi-automated prescription of scanning parameters which is optimally suited for repeated re-scanning of the same patient, thereby making use of characteristic anatomical properties of the examined individual.

In accordance with the present invention, a method for prescription of scanning parameters of the type specified above is disclosed, wherein the aforementioned problems and drawbacks are avoided by the computation of the current orientation and location of the object relative to its orientation and location during the previous examination by matching the current reference scan image data with the image data of a previous reference scan, the current examination scanning parameters being calculated by adjusting the scanning parameters of the previous examination in accordance with the relative orientation and location of the object during the current examination.

The present invention enables to perform tomographic scanning with the relation between the geometries of the patient and the imaging planes being equal at each repeated examination session. This is achieved by the registration of reference scan image data which is employed to establish a well defined initial scanning geometry. The reference scan is performed as well during the previous as during the current examination with sufficient anatomical coverage to safely assess the orientation and location of the patient in the scanner. The previous angulation and off-center parameters, which were for example determined when the examination was manually planned at the first session, are reestablished at the current examination by comparison of the previous and the current reference scan image data. For the method of the invention, which in practice has to be implemented on a computer, it is needed to have the digital data of the previous examinations available. This requirement can easily be met by adding the necessary image data to the corresponding entry of a patient database as it is well known in the art.

The method of the present invention is simple, fast, precise and very robust in terms of assessing the orientation and location of the examined object in the scanner because it makes use of the characteristic features of the same object which has already been examined at a previous session. No difficult model reconstruction, as it is described in the above cited U.S. patent, is needed. It is particularly advantageous that the investigation of the relative geometric data can be performed easily and fast by taking low resolution images of a volume of interest which has been defined during the previous examination of the patient. Once the relative orientation and location of the patient is established, the new scanning parameters are prescribed by simply adjusting the corresponding parameters of the previous examination accordingly. The previous scanning parameters can be made available most easily by including them into the patient database entry.

With the method of the invention it is useful if the matching of the current and previous reference scan image data is performed by identification of three or more corresponding landmark points in the scan volumes of both the current and previous reference scans.

Three landmark points in space, which might for example represent the location of characteristic anatomic features, span a plane whose orientation (angulation) relative to the coordinates of the scan volume of the reference scan is known for both the previous and the current examination. The off-centers are known for both, too. From the previous data, including the coordinates of the landmark points relative to the reference scan volume and the orientations and locations of the examination imaging planes relative to the geometry of the reference scan, the scanning parameters for the subsequent current examination are derived by adding the relative off-centers and angulations of the plane spanned by the landmark points of the current reference scan in order to obtain equal imaging planes for the current examination. Optionally more than three landmark points might be identified in order to increase the accuracy of the method. Three landmark points are sufficient to compensate for translations and rotations of the examined object between subsequent examinations. If more than three landmark points are employed also stretching and bending is taken into account.

The above described identification of landmark points can be carried out either manually by an operator of the scanning device or automatically by means of an appropriate image recognition or pattern matching algorithm. According to the method of the present invention, the matching of the current and previous reference scan image data is in this case performed by recognition of characteristic features in the images of both the current and the previous reference scans.

It is advantageous to carry out the matching of the current and previous reference scan image data by identification of landmark points, because in this case the scanning parameters of the previous and current reference scans do not necessarily have to be identical. When using anatomical landmarks, variations in the field of view or in the contrast of the images can easily be dealt with. It is also possible to employ any diagnostic anatomical scan as reference scan images for the identification of the anatomical landmarks as long as the position of the imaging planes relative to the geometry of the landmark points is known. Therefore it is useful if the relative geometry of the examination imaging planes and the anatomical landmark points is also stored in the patient database, thereby enabling the scanning parameters to be calculated automatically once the landmark points are identified in the current reference image data.

As an useful alternative, by which the involvement of a human operator is completely eliminated, it is also possible to perform the matching of the current and previous reference scan image data by finding a geometric transformation that minimizes the differences between the two images. This can be done by application of standard algorithms which iteratively compute the parameters of the geometric transformation until the difference between the two reference images is minimal. The resulting geometric transformation is defined by rotation angles and a translation vector representing the relative orientation and location of the patient with respect to the previous examination. These data can directly be used to compute the scanning parameters of the current examination by applying the same geometric transformation to the scanning parameters of the previous examination.

A computer program adapted for carrying out the method of the present invention employs a matching algorithm which processes the image data of the current reference scan, thereby extracting geometric data defining the current orientation and location of the examined object in the scanner, and further relating the current geometric data of the examined object to the corresponding geometric data of the same object during a previous examination. This matching algorithm computes the current orientation and location of the object relative to its orientation and location during the previous examination by matching the current reference scan image data with the image data of a corresponding previous reference scan, the current examination scanning parameters being calculated by adjusting the scanning parameters of the previous examination in accordance with the relative orientation and location of the object during the current examination.

Such a computer program can advantageously be implemented on any common computer hardware which is presently in clinical use for the control of tomographic imaging apparatus, such as for example MRI or CT scanners. The computer program can be provided on suitable data carriers, such as CD-ROM or diskette. Alternatively, it can also be downloaded by a user from an internet server.

For a practical implementation of such a computer program, both the current and the previous reference images are presented to the operator by means of a computer display device, thereby enabling the operator to perform the matching of the current and the previous reference image data interactively by identifying three or more corresponding landmark points in the scan volumes of both the current and previous reference scans.

The computer program can for example provide a user interface with multiple viewports to present different three dimensional views of the examined object to the operator. Both the previous and the current reference scan images are displayed in different viewports. A very intuitive method for facilitating the matching of the previous and the current reference scan images is to display slices through different anatomical landmarks in the viewports. The orientations and locations of these slices can be interactively manipulated by the user. The matching procedure is completed when the user decides that corresponding slices which are displayed in the different viewports have the same locations and orientations relative to the position of the examined object in the images of both the previous and the current reference scans.

In order to obtain maximum accuracy, it is furthermore advantageous if the matching of the current and previous reference scan image data is refined by automatic recognition of characteristic features in the images of both the current and the previous reference scans and/or by finding a geometric transformation that minimizes the differences between the two images. Thus an optimal result is obtained by alternately applying an interactive, user-controlled and a fully automatic matching procedure. It is possible to start the matching of the previous with the current reference scan image data with either a user-controlled or an automatic matching method. In case of large differences between the previous and the current examination, which can for example be due to the progress of the disease or to the resection of a tumor, the automatic registration of the position of the patient in the scanner may fail. In such situations the user may decide to start the matching procedure interactively and subsequently refine the localization by an automatic matching algorithm.

Interactive matching by finding a geometric transformation that minimizes the differences between the two images can be implemented by the visualization of subtraction images. If the same scanning parameters are employed for both the previous and the current reference scans, the matching can be indicated to the user by displaying a difference image of the previous and the current reference scans. The optimal transformation is found if the difference image shows a more or less homogeneous minimum intensity.

It is possible to incorporate the method of the present invention in dedicated tomographic imaging apparatus, such as MRI or CT devices, comprising a scanner and a computer, wherein the scanner comprises means for generating tomographic images according to scanning parameters being prescribed by the computer, and wherein the computer comprises a memory and a program control which operates according to the method of the present invention.

### DRAWINGS

The following drawings disclose preferred embodiments of the present invention. It should be understood, however, that the drawings are designed for the purpose of illustration only and not as a definition of the limits of the invention.

In the drawings

FIG. 1 shows a representation of the method of the present invention as a block diagram;

FIG. 2 shows a magnetic resonance system adapted to operate according to the method of the present invention.

### DESCRIPTION

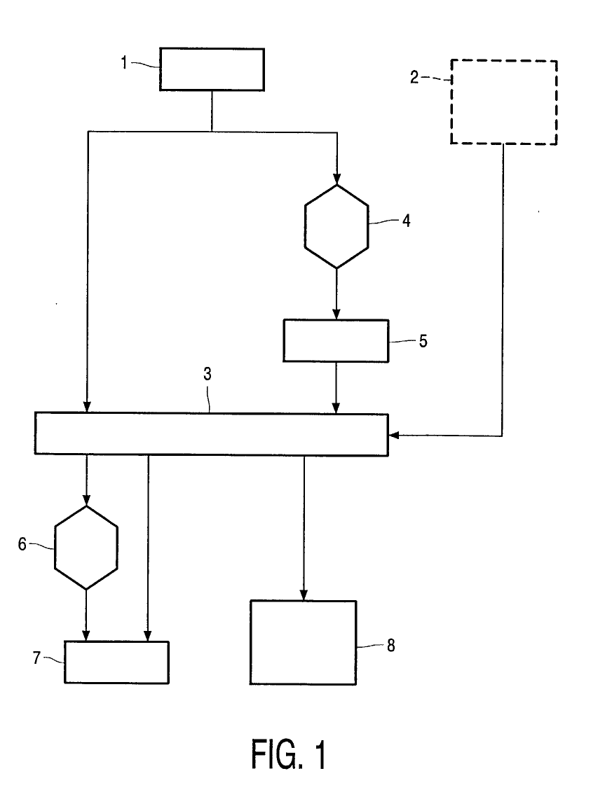

FIG. 1 represents the workflow of a typical implementation of the present invention. It starts with the registration of a survey scan image 1. For this purpose it is sufficient to take low resolution images of a volume of interest which is selected to allow for a reliable assessment of the position of the patient in the scanner. After step 1 it is either possible to start directly with the extraction of the required geometric data by taking the survey scan 1 as current reference scan image data in the sense of the present invention. In this case, the image data from a previous examination 2, which is stored in the patient database entry, is used to perform the matching of the current reference scan image with the image data of the corresponding previous reference scan in step 3. As an alternative, the image data of the survey scan 1 is transferred to a plan scan tool which is used by the operator of the tomographic scanner in step 4 to interactively plan the subsequent scanning of the patient. In step 5, either a full anatomical examination scan or just a short reference scan is initiated in accordance with the prescription of the operator in step 4. Thereafter, the actual matching of the previous and the current reference image data is carried out in step 3. The current reference scan image data can either be the survey image of step 1 or at least parts of the image data generated in step 5. The corresponding previous reference scan image data 2, which are provided in digital form by the patient database, are selected automatically.

There are different possibilities of computing the current orientation and location of the examined object relative to its orientation and location during the previous examination. As described above, both the current and the previous reference scan images can be presented to the operator who performs the matching interactively by identifying three or more corresponding landmark points in the respective images. Additionally, the matching of the reference scan image data can be refined by automatic recognition algorithms or by finding a geometric transformation that minimizes the differences between the previous and the current images. Depending on the scanning parameters of the reference scans it might be necessary to interpolate the image data in accordance with the scanning parameters of the previous examination session 2 before the actual matching of corresponding images can be carried out.

Once the relative orientation and location of the patient with respect to the previous examination 2 is settled, also the calculation of the current examination scanning parameters is performed in step 3. The corresponding scanning parameters of the previous examination 2 are again extracted from the patient database and adjusted in accordance with the relative position of the patient during the current examination.

Thereafter, the angulations and off-center parameters which have been computed in step 3 can be presented to the operator by means of a plan scan tool in step 6. The operator can check and confirm the results of the matching procedure. He is also enabled to further adjust the scanning parameters manually before the actual examination scanning is initiated in step 7. It is also possible to start the scanning procedure 7 immediately without any involvement of the operator. If a full anatomical scan was performed in step 5, redundant scanning is avoided by computing “re-sliced” images based on the image data in step 8 in accordance with the image plane orientations and locations which have been computed in step 3. This allows for the application of the method of the invention as a mere post-processing of conventionally registered image data.

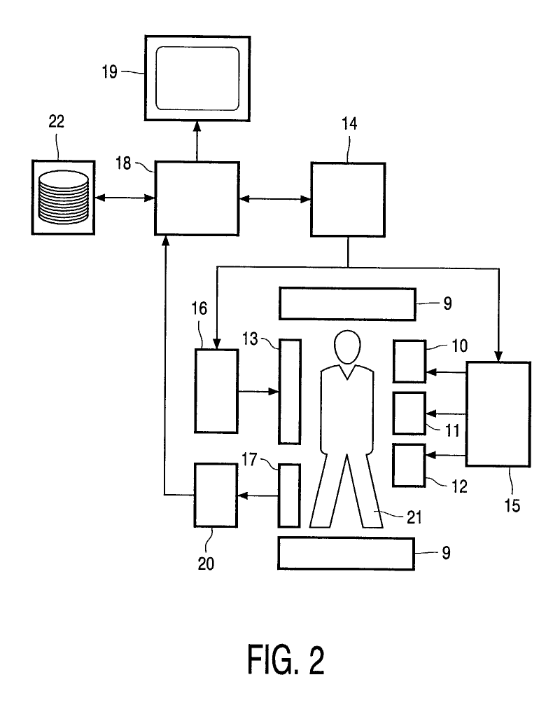

A magnetic resonance system as shown in FIG. 2 is suitable for carrying out the method of the invention. It includes a coil 9 for generating a steady, uniform magnetic field, gradient coils 10, 11 and 12 for generating gradient pulses in the x, the y and the z direction, and an RF transmission coil 13. The temporal succession of the gradient pulses is controlled by means of a control unit 14 which communicates with the gradient coils 10, 11 and 12 via a gradient amplifier 15. Furthermore, the control unit 14 is connected to the RF transmission coil 13 via an RF transmission amplifier 16, so that powerful RF pulses can be generated. The MR signals, which are excited by the RF pulses, are registered by a RF receiving coil 17. The system also includes a reconstruction unit in the form of a microcomputer 18 as well as a visualization unit 19 which may be a graphic monitor. The spin resonance signals, which are registered by the RF receiving coil 17 are demodulated and amplified by a receiver unit 20. In the reconstruction unit 18 the spin resonance signals are subjected to Fourier analysis in order to generate images of regions of interest of a patient 21. The method of the invention is implemented as a computer program in the reconstruction unit 18. The reconstructed current reference images are processed according to the invention. Therefore, the previous imaging data, which is required for the matching procedure, is included from a patient database server 22. The reconstruction unit 18 transfers the scanning parameters, which are computed in accordance with the present invention, to the control unit 14, which initiates the desired re-scanning procedure of the patient 21.

The invention has been described with reference to the preferred embodiment. Obviously, modifications and alterations will occur to others upon reading and understanding the preceding detailed description. It is intended that the invention be construed as including all such modifications and alterations insofar as they come within the scope of the appended claims or the equivalents thereof.

What is claimed is:

1. Method for prescription of scanning parameters determining the orientation and location of tomographic imaging planes, wherein a current reference scan (1) of an object is performed, the image data of the reference scan being analyzed, thereby extracting geometric data defining the current orientation and location of the examined object in the scanner, and wherein scanning parameters for one or more current examination scans (7) are computed by relating the current geometric data of the examined object to the corresponding geometric data of the same object during a previous examination (2), wherein the current orientation and location of the object relative to its orientation and location during the previous examination (2) is computed by matching (3) the current reference scan image data with the image data of a previous reference scan, the current examination scanning parameters being calculated by adjusting the scanning parameters of the previous examination (2) in accordance with the relative orientation and location of the object during the current examination.

2. Method of claim 1, wherein the matching of the current and previous reference scan image data is performed by identification of three or more corresponding landmark points in the scan volumes of both the current and previous reference scans.

3. Method of claim 1, wherein the matching of the current and previous reference scan image data is performed by recognition of characteristic features in the images of both the current and the previous reference scans.

4. Method of claim 1, wherein the matching of the current and previous reference scan image data is performed by finding a geometric transformation that minimizes the differences between the two images.

5. Computer program which prescribes the parameters defining the location and orientation of tomographic imaging planes for one or more current examination scans by processing the image data of a current reference scan, thereby extracting geometric data defining the current orientation and location of the examined object in the scanner, and further relating the current geometric data of the examined object to the corresponding geometric data of the same object during a previous examination, wherein the current orientation and location of the object relative to its orientation and location during the previous examination is computed by matching the current reference scan image data with the image data of a corresponding previous reference scan, the current examination scanning parameters being calculated by adjusting the scanning parameters of the previous examination in accordance with the relative orientation and location of the object during the current examination.

6. Computer program of claim 5, wherein both the current and the previous reference images are presented to a user by means of a computer display device, thereby enabling the user to perform the matching of the current and the previous reference image data interactively by identifying three or more corresponding landmark points in the scan volumes of both the current and previous reference scans.

7. Computer program of claim 6, wherein the matching of the current and previous reference scan image data is refined by automatic recognition of characteristic features in the images of both the current and the previous reference scans and/or by finding a geometric transformation that minimizes the differences between the two images.

8. Tomographic imaging apparatus comprising a scanner and a computer, wherein the scanner comprises means for generating tomographic images according to scanning parameters being prescribed by the computer, and wherein the computer comprises a memory and a program control wherein a current reference scan (1) of an object is performed, the image data of the reference scan being analyzed, thereby extracting geometric data defining the current orientation and location of the examined object in the scanner, and wherein scanning parameters for one or more current examination scans (7) are computed by relating the current geometric data of the examined object to the corresponding geometric data of the same object during a previous examination (2), wherein the current orientation and location of the object relative to its orientation and location during the previous examination (2) is computed by matching (3) the current reference scan image data with the image data of a previous reference scan, the current examination scanning parameters being calculated by adjusting the scanning parameters of the previous examination (2) in accordance with the relative orientation and location of the object during the current examination.
